(19) United States
(12) Patent Application Publication
(10) Pub. No.: US 2024/0277413 A1
(43) Pub. Date: Aug. 22, 2024

(54) DETERMINING A POSITION FOR AN IMPLANT RELATIVE TO A BONE BASED ON BONE REMODELING DATA

(71) Applicant: Think Surgical, Inc., Fremont, CA (US)

(72) Inventor: Stuart Simpson, Fremont, CA (US)

(73) Assignee: Think Surgical, Inc., Fremont, CA (US)

(21) Appl. No.: 18/442,265

(22) Filed: Feb. 15, 2024

Related U.S. Application Data
(60) Provisional application No. 63/446,764, filed on Feb. 17, 2023.

(51) Int. Cl. A61B 34/10

(52) U.S. Cl. CPC A61B 34/10; A61B 2034/104; A61B 2034/105; A61B 2034/107

(57) ABSTRACT

A computer-assisted method is provided for determining a placement position for an implant relative to a bone. According to the method, a force profile exerted on a bone is calculated from bone density data and bone architecture data. A position and orientation (POSE) for an implant in relation to the bone based is then determined, at least in part, on the calculated force profile. A computing system is provided that includes a computer having a processor and non-transient memory programmed with operating planning software that when executed by the processor is configured to: calculate a force profile exerted on a bone from bone density data and bone architecture data and determine a position and orientation (POSE) for an implant in relation to the bone based, at least in part, on the calculated force profile.

### RELATED APPLICATIONS

This application claims priority benefit of U.S. Provisional Application Ser. No. 63/446,764 filed Feb. 17, 2023; the contents of which are hereby incorporated by reference.

### TECHNICAL FIELD

The present invention generally relates to computer-assisted orthopedic surgery, and more particularly to a system and method for determining a position for an implant relative to a bone based on bone remodeling data.

### BACKGROUND

Throughout a lifetime, bones and joints become damaged and worn through normal use, disease, and traumatic events. Arthritis is a leading cause of joint damage, which can cause cartilage degradation, pain, swelling, stiffness, and bone loss overtime. If the pain associated with the dysfunctional joint is not alleviated by less-invasive therapies, the joint may need to be replaced with a procedure called total joint arthroplasty (TJR). TJR is an orthopedic surgical procedure in which the typically worn articular surfaces of the joint are replaced with prosthetic components, or implants. TJR typically requires the removal of the articular cartilage of the joint including a varying amount of bone. This cartilage and bone are then replaced with synthetic implants, typically metal and plastic, which form the new synthetic joint surfaces.

In conventional joint replacement surgeries including knee joints and total hip arthroplasty (THA), a surgeon may use planar x-rays to plan the placement for an implant relative to the bone. Implant templates are overlaid on the radiographs to determine the proper implant size for an implant to fit a patient's bone. For example, in an operating room (OR) for a THA procedure, the surgeon aims to ream an acetabulum to achieve a cup implant placement of 40° (+10°) in inclination and 15° (+10°) in anteversion. These clinically established alignment targets are known as the Lewinnek safe zone and have been shown to provide the best surface area for a ball joint of a femoral implant, reduced rates of dislocation, and are less likely to cause impingement. However, the surgeon typically has a difficult time aligning a reamer to achieve these Lewinnek safe zone alignment targets in three dimensions with the complex geometries present. In addition, the +10° acceptable deviation range of the Lewinnek safe zone is significantly broad, where some patients may have better clinical outcomes with the implant placed on one end of the deviation range (e.g., −10°) with respect to the bone compared to other patient's that may fair better on the other end of the deviation range (e.g. +10°). It is further contemplated that some patients may have better clinical outcomes with an implant placed outside the Lewinnek safe zone with respect to the bone, however there is currently no surgical planning method or intra-operative technique to determine which patients may benefit therefrom.

In addition, in the last 10 years, there has been increasing acceptance that the natural position and orientation (POSE) of a patient's hip changes when laying down on a surgical table relative to a standing position in which the joint supports the patient's upper body weight. Geometrically, standing weight loading and biomechanics of the joint results in a pelvic tilt, either forward or backward, compared to an unweighted laying down position. As a result, surgeons have adopted using different X-ray techniques and/or X-ray views to plan the THA procedure, such as X-ray views of the patient laying down, sitting down, and standing up to account for changes in the pelvic POSE as a function of biomechanics and joint weight loading. The surgeon can then plane the placement for the implant in the Lewinnek safe zone for each X-ray view. However, as discussed above, the Lewinnek safe zone has a significantly broad deviation range. In addition, X-ray imaging affords limited information about the three-dimensional (3-D) geometry of the bone and the bone quality.

To further assist in surgical planning, advanced planning software has been developed to plan the placement for an implant relative to a bone using 3-D bone models and 3-D implant models. The planning software generates 3-D bone models of the patient's bone using imaging data such as computed tomography (CT) or magnetic resonance imaging (MRI) data. Models of the implants are provided in the planning software where a user may virtually manipulate the implant models relative to the bone models to determine: (i) the best fit, fill, and alignment for a given implant; (ii) an optimal implant from a catalog of premade implants; and/or (iii) a design of a custom implant relative to the bone, all according to clinically established alignment goals. However, these alignment goals (e.g., Lewinnek safe zone) are standardized and generally have significantly broad deviation ranges, as noted above. Therefore, even with the geometry data provided by the generation of a 3-D bone model, it is impossible to know if there is a more optimal location for the implant with respect to the bone that is within the deviation range, or even outside the deviation range, for a given patient. What the clinically established alignment goals fail to particularly address is the differences in the natural motion (e.g., motion of the joint while walking, running, and performing routine activities) of each individual patient's joint. Each individual patient's joint moves in a unique manner that results in different loading conditions or imposed forces between the bones of the patient's joint. As a result, following a THA or other type of implant procedure, one patient may impose different forces on their implants compared to another patient even if both implants are aligned to the same clinically established alignment goal. This unaccounted-for difference in loading or applied forces may cause one patient's implant(s) to fail as compared to another patient having the same procedure, everything else being equal.

Thus, there exists a need for a system and method to determine a position for an implant relative to a bone that accounts for the unique natural forces within an individual patient's joint. There further exists a need for determining the natural forces within an individual patient's joint to plan a position for an implant relative to the bone.

### SUMMARY OF THE INVENTION

A computer-assisted method is provided for determining a placement position for an implant relative to a bone. According to the method, a force profile exerted on a bone is calculated from bone density data and bone architecture data. A position and orientation (POSE) for an implant in relation to the bone based is then determined, at least in part, on the calculated force profile.

A computing system is provided that includes a computer having a processor and non-transient memory programmed with operating planning software that when executed by the processor is configured to: calculate a force profile exerted on a bone from bone density data and bone architecture data and determine a position and orientation (POSE) for an implant in relation to the bone based, at least in part, on the calculated force profile.

### BRIEF DESCRIPTION OF THE DRAWINGS

The present invention is further detailed with respect to the following drawings that are intended to show certain aspects of the present of invention, but should not be construed as limit on the practice of the invention, wherein:

FIG. 1 is a general view of a pelvis and a femur of a patient, where the acetabulum of the pelvic bone couples with the femoral head of the femur to form a hip joint;

FIG. 2 depicts a 3-D bone model of an acetabulum derived from computed tomography (CT) data in accordance with embodiments of the invention;

FIGS. 3A and 3B depict a profile view of a first patient's (patient ‘A’) acetabulum and a profile view of a second patient's (patient ‘B’) acetabulum, respectively as derived from the CT data, where the architecture of the bone at different locations of the acetabulum is shown, in accordance with embodiments of the invention;

FIGS. 4A and 4B depict calculated force profiles for patient ‘A’, and for patient ‘B’ where each force profile includes a series of lines, where the magnitude of the line correlates to the magnitude of the force in accordance with embodiments of the invention;

FIG. 5 depicts a plurality of voxels with corresponding the Hounsfield Units (HU) representing density values of the bone at different locations on the femoral head in accordance with embodiments of the invention;

FIG. 6 depicts bone architecture at various locations on the femoral head;

FIG. 7 depicts a calculated force profile on the femoral head shown as a series of lines, where the magnitude of the lines correlates to the magnitude of the imposed forces in accordance with embodiments of the invention;

FIG. 8 depicts an embodiment of a planning workstation for implementing the method of the invention; and

FIGS. 9A and 9B depict a display of a surgical plan for patient ‘A’ and ‘B’ as displayed on a graphical user interface (GUI) that show an acetabulum, a cup implant, and calculated force profile, the surgical plan generated by software to position the cup implant with respect to the acetabulum such that the forces imposed on the acetabulum by the cup implant following the THA procedure will substantially match the force profile.

### DETAILED DESCRIPTION

The present invention has utility as a system and method for determining a position for an implant relative to a bone based on bone remodeling data. Embodiments of the invention take into account the forces exerted on a bone within a joint when a person moves or changes their body position to optimize the placement of a joint replacement. A bone will remodel according to the forces imposed on it, known commonly in the field as Wolf's Law. In some inventive embodiments, a 3-D bone model and CT data is used to determine the bone remodeling data, and to identify an optimal position for joint implants, such as hip implants for each individual patient. While the invention is depicted herein in the context of a hip joint, it is appreciated that the present invention is applicable to a variety of anatomical joint replacements that also include shoulder, ankle, knee, finger, toe, elbow; as well in dental prothesis and ocular socket refinishing to receive an implant. The CT data and bone model of a specific patient is used to determine the spatial distribution of the imposed forces between the bones of a joint. With the CT data and 3-D bone model, the optimal implant placement for each patient can be determined to create a desired force profile, such as a same force profile (i.e., place the implants in a position that will keep the same force profile). With use of embodiments of the invention, surgical implant planning is now based on how the patient's joint naturally moved as determined by the remodeling of each patient's bone. It is appreciated that in some embodiments of the present invention, the implant positioning may be surgeon-selected to be a hybrid of different implant placement strategies. Some factors relevant to a hybrid implant placement strategy include the patient activity level, the condition of other joints of the patient, patient body mass index, specific joint stressors imposed by specific sporting activities, or a combination of any of the aforementioned.

The present invention will now be described with reference to the following embodiments. As is apparent by these descriptions, this invention can be embodied in different forms and should not be construed as limited to the embodiments set forth herein. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations, and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references mentioned herein are incorporated by reference in their entirety.

The following description provides examples related to hip joint replacement; however, it should be appreciated that the embodiments described herein are readily adapted for use in a myriad of applications where it is desirous to ascertain the forces exerted on a joint interface during natural movement to optimize joint replacements in other portions of the body.

It is to be understood that in instances where a range of values are provided that the range is intended to encompass not only the end point values of the range but also intermediate values of the range as explicitly being included within the range and varying by the last significant figure of the range. By way of example, a recited range of from 1 to 4 is intended to include 1-2, 1-3, 2-4, 3-4, and 1-4.

### Definitions

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

Also, as used herein, “and/or” refers to and encompasses any and all possible combinations of one or more of the associated listed items, as well as the lack of combinations when interpreted in the alternative (“or”).

As used herein, the term “bone data” refers to data related to one or more bones. The bone data may be determined: (i) prior to making modifications (e.g., bone cuts, insertion of a pin or screw, etc.) to one or more bones; and/or (ii) determined after one or more modifications have been made to a bone or bone piece/fragment. The bone data may be collected, generated, and/or determined preoperatively, intraoperatively, or a combination of pre-operatively and intraoperatively. The bone data may include: the shapes of the one or more bones; the sizes of the one or more bones; angles and axes associated with the one or more bones (e.g., epicondylar axis of the femoral epicondyles, longitudinal axis of the femur, the mechanical axis of the femur); angles and axes associated with two or more bones relative to one another (e.g., the mechanical axis of the knee); anatomical landmarks associated with the one or more bones (e.g., femoral head center, knee center, ankle center, tibial tuberosity, epicondyles, most distal portion of the femoral condyles, most proximal portion of the femoral condyles); bone density data; bone architecture data; and stress/loading conditions of the bone(s). By way of example, the bone data may include one or more of the following: an image data set of one or more bones (e.g., an image data set acquired via fluoroscopy, computed tomography (CT), magnetic resonance imaging (MRI), ultrasound, other x-ray modalities, laser scan, etc.); three-dimensional (3-D) bone models, which may include a virtual generic 3-D model of the bone, a physical 3-D model of the bone, a virtual patient-specific 3-D model of the bone generated from an image data set of the bone; and a set of data collected directly on the bone intra-operatively commonly used with imageless computer-assisted surgical devices (e.g., laser scanning the bone, collecting a cloud of points on the bone with a digitizer, “painting” the bone with a digitizer).

As used herein, the terms “computer-assisted surgical device” and “CAS device” refer to devices used in surgical procedures that are at least in part assisted by one or more computers. Examples of CAS devices illustratively include tracked/navigated instruments and surgical robots. Examples of a surgical robot illustratively include robotic hand-held devices, serial-chain robots, bone mounted robots, parallel robots, or master-slave robots, as described in U.S. Pat. Nos. 5,086,401; 6,757,582; 7,206,626; 8,876,830; 8,961,536; 9,707,043; and 11,457,980; which patents and patent application are incorporated herein by reference. The surgical robot may be active (e.g., automatic/autonomous control), semi-active (e.g., a combination of automatic and manual control), haptic (e.g., tactile, force, and/or auditory feedback), and/or provide power control (e.g., turning a robot or a part thereof on and off). It should be appreciated that the terms “robot” and “robotic” are used interchangeably herein. The terms “computer-assisted surgical system” and “CAS system” refer to a system comprising at least one CAS device and may further include additional computers, software, devices or instruments. An example of a CAS system may include: i) a CAS device and software used by the CAS device (e.g., cutting instructions, pre-operative bone data); ii) a CAS device and software used with a CAS device (e.g., surgical planning software); iii) one or more CAS devices (e.g., a surgical robot); iv) a combination of i), ii), and iii); and iv) any of the aforementioned with additional devices or software (e.g., a tracking system, tracked/navigated instruments, tracking arrays, bone pins, rongeur, an oscillating saw, a rotary drill, manual cutting guides, manual cutting blocks, manual cutting jigs, etc.).

Also referenced herein is a “surgical plan”. A surgical plan is generated using planning software. The surgical plan may be generated pre-operatively, intra-operatively, or pre-operatively and then modified intra-operatively. The planning software may be used to plan the location for an implant with respect to a bone and/or plan a location to make one or more modifications (e.g., bone cuts, location for inserting bone pins) to the bone. The planning software may include various software tools and widgets for planning the surgical procedure. This may include, for example, planning: (i) a location for a 3-D implant model with respect to a 3-D bone model to define a location for the implant with respect to the bone; (ii) a location for one or more cuts to be made relative to a 3-D bone model to define the locations for one or more cuts to be made on the bone, and/or (ii) one or more locations on a 3-D bone model for inserting hardware (e.g., bone pins, screws) to define locations for one or more virtual references (e.g., a virtual plane, virtual boundary, a virtual axis) with respect to the bone, where a CAS device is directed to align an end-effector (e.g., the hardware, a burr, end-mill, drill bit) with the location of the virtual reference(s) registered to the bone.

As used herein, the term “registration” refers to: the determination of the spatial relationship between two or more objects; the determining of a coordinate transformation between two or more coordinate systems associated with those objects; the mapping of an object onto another object; and a combination thereof. Examples of objects routinely registered in an operating room (OR) illustratively include: CAS systems/devices; anatomy (e.g., bone); bone data (e.g., 3-D virtual bone models); a surgical plan (e.g., location of virtual planes defined relative to bone data, cutting instructions defined relative to bone data); and any external landmarks (e.g., a tracking array affixed to a bone, an anatomical landmark, a designated point/feature on a bone, etc.) associated with the bone (if such landmarks exist). Methods of registration known in the art are described in U.S. Pat. Nos. 6,033,415; 8,010,177; 8,036,441; and 8,287,522; and 10,537,388. In particular embodiments with orthopedic procedures, the registration procedure relies on the manual collection of several points (i.e., point-to-point, point-to-surface) on the bone using a tracked digitizer where the surgeon is prompted to collect several points on the bone that are readily mapped to corresponding points or surfaces on a 3-D bone model. The points collected from the surface of a bone with the digitizer may be matched using iterative closest point (ICP) algorithms to generate a transformation matrix. This transformation matrix and various other transformation matrices provides the correspondence between: (i) one or more targets or boundaries defined in a surgical plan (e.g., a pre-defined location for a targeted virtual plane that was defined with respect to bone data, a pre-defined location of cutting instructions that was defined with respect to bone data); (ii) the coordinate system of a tracking array affixed to the bone (if present); (iii) a CAS device (e.g., the base coordinate system of the CAS device, or a coordinate system of a tracking array affixed to the CAS device and, if needed, calibration data and/or kinematic data that define the location of an end-effector relative to the tracking array); and any other coordinate system or object required to perform the procedure. In other embodiments, the registration is performed using image or imageless registration.

In certain embodiments of the present invention, the bone remodeling data may include bone density data and bone architecture data, which are used to calculate a force profile on a bone. The force profile indicates the forces that were imposed at a plurality of locations on the bone within a joint. The bone density data may include bone density values at a plurality of locations on the bone, and the bone architecture data may include the architecture of the bone at a plurality of locations on the bone. The bone density data and bone architecture data indicate how the bone remodeled within the joint as a result of the forces imposed on the bone through the natural motion of the joint. A force profile is readily calculated using the bone density data and bone architecture data with techniques known in the art, such as finite element analysis or machine learning. Methods for determining a force profile on a bone using the aforementioned data is described in: Christen, Patrik, et al. “Determination of hip-joint loading patterns of living and extinct mammals using an inverse Wolff's law approach.” Biomechanics and modeling in mechanobiology 14.2 (2015): 427-432; Christen, Patrik, et al. “Bone morphology allows estimation of loading history in a murine model of bone adaptation.” Biomechanics and Modeling in Mechanobiology 11.3 (2012): 483-492; Zadpoor, Amir Abbas, Gianni Campoli, and Harrie Weinans. “Neural network prediction of load from the morphology of trabecular bone.” Applied Mathematical Modelling 37.7 (2013): 5260-5276. The “forces” described herein may have a magnitude component or both a magnitude component and a direction component (e.g., vector quantity). A “load” refers to a force exerted on a surface or body. A “force profile” may also be referred to herein as a “load profile” and generally refers to a profile of forces exerted on a surface or body.

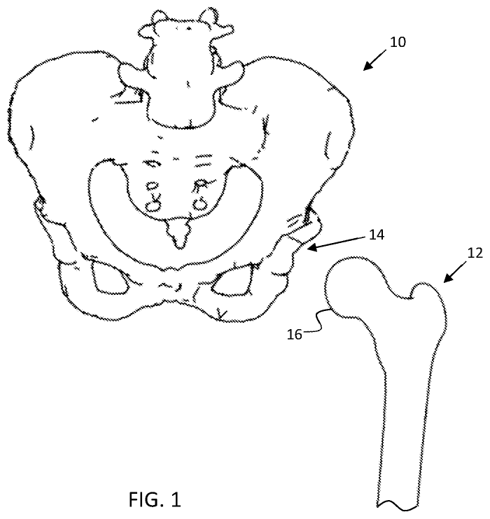

Referring now to the figures, FIG. 1 depicts a pelvis 10 and a femur 12 of a patient. The pelvis 10 includes the acetabulum 14, which is a socket in the pelvis 10 that couples with the femoral head 16 of the femur 12 to form the hip joint.

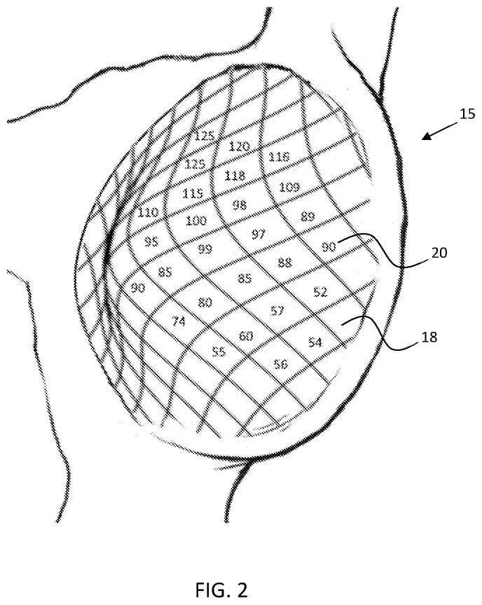

FIG. 2 depicts bone data (e.g., 3-D bone model) of an acetabulum 15 generated from CT data. The CT data includes a plurality of voxels 18, each voxel contains density data of the bone at the location of that voxel, known as Hounsfield Units (HUs) 20. Each voxel may have a HU associated with it, as shown by the numbers in the voxel grid. The HUs may represent relative bone density or the HUs may be converted into absolute or true density values using techniques know in the art, such as those described in U.S. Pat. No. 10,631,934 assigned to the assignee of the present application. For example, an object having different materials, each material with a different and known density, may be scanned with the bone to provide a basis for making the conversions to absolute density. Areas of denser bone suggest how the bone remodeled to support areas of greater forces experienced on the bone per Wolf's Law. The profile of the bone density may therefore provide information about the forces imposed on the bone from the natural motion of the patient's joint. The bone density values and profile may then be used according to the present invention as an input to calculate a force profile to assist in determining (e.g., planning) a position for an implant relative to the bone.

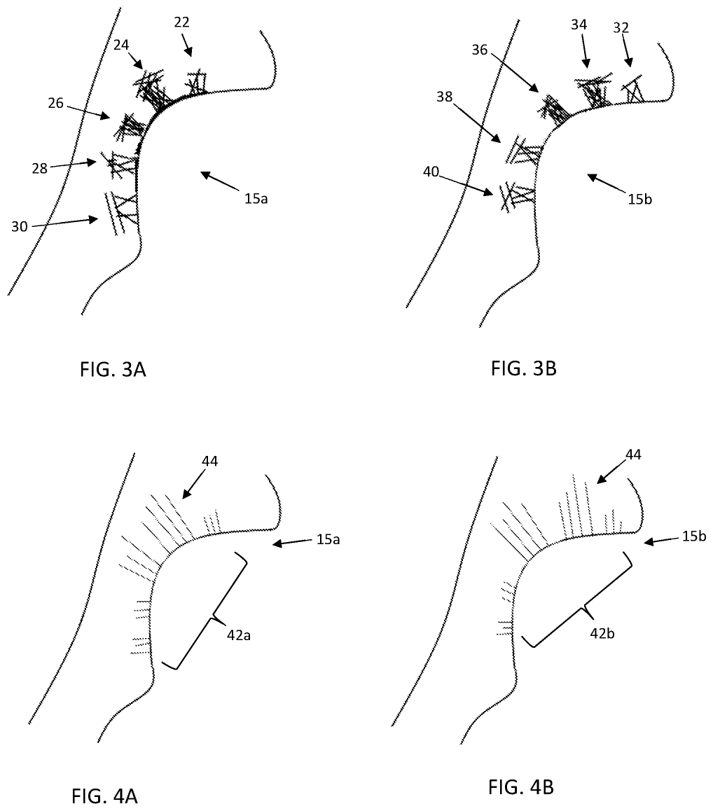

FIG. 3A depicts a profile view of acetabulum bone data 15a from a first patient (patient ‘A’) and FIG. 3B depicts a profile view of acetabulum bone data 15b from a second patient (patient ‘B’). The architecture of the bone at different locations of the acetabulum is shown, which may also be derived from the CT data. Other imaging may be performed to obtain a more detailed view of the architecture, such as quantitative CT imaging or micro-CT, however, standard CT data may be sufficient in most cases. In FIG. 3A, patient ‘A’ has a more compact trabecular bone architecture, and boney structures more normal to the acetabulum surface, at locations 24 and 26 compared to locations 22, 28, and 30 that are less so. In FIG. 3B, patient ‘B’ has a more compact trabecular bone structure, and boney structures more normal to the acetabulum surface, at locations 34 and 36 compared to locations 32, 28, and 40 that are less so. The primary difference between the bone architecture of patient ‘A’ versus patient ‘B’ is patient ‘B’ has a more compact trabecular architecture that is shifted towards the top of the acetabulum compared to patient ‘A’. This suggests a difference in the bone remodeling profile between patient ‘A’ and patient ‘B’ and therefore a difference in the natural motion of the joint for patient ‘B’ compared to patient ‘A’. The location, compactness, and orientation of the bone architecture may be used as an input to calculate a force profile to assist in planning the position for an implant relative to the bone.

FIG. 4A depicts the calculated force profile 42a for patient ‘A’, and FIG. 4B depicts the calculated force profile 42b for patient ‘B’. Each force profile (42a, 42b) includes a series of lines 44, where the magnitude of the line correlates to the magnitude of the force. The force profile may alternatively be generated as a heat map, a matrix of values, or other graphical technique. The force profile may be viewed in 2-D or 3-D, where the user may have options to select different views (e.g., coronal, sagittal, or axial view) as well as select different slices on the 2-D image data or 3-D bone model to view the force profile at the selected slice. Again, the force profile 42b for patient ‘B’ indicates higher forces at the top of the acetabulum compared to the force profile 42a for patient ‘A’ which is toward the center. The force profile indicates how the acetabulum portion of the pelvic bone remodeled under the loading conditions for the patient's joint and is therefore also indicative of the natural motion of the patient's joint and the interaction between femoral head 16 and the acetabulum 14 in the case of a hip joint. It is appreciated that other skeletal joints including the knee, shoulder, and elbow may also be modeled in a similar manner.

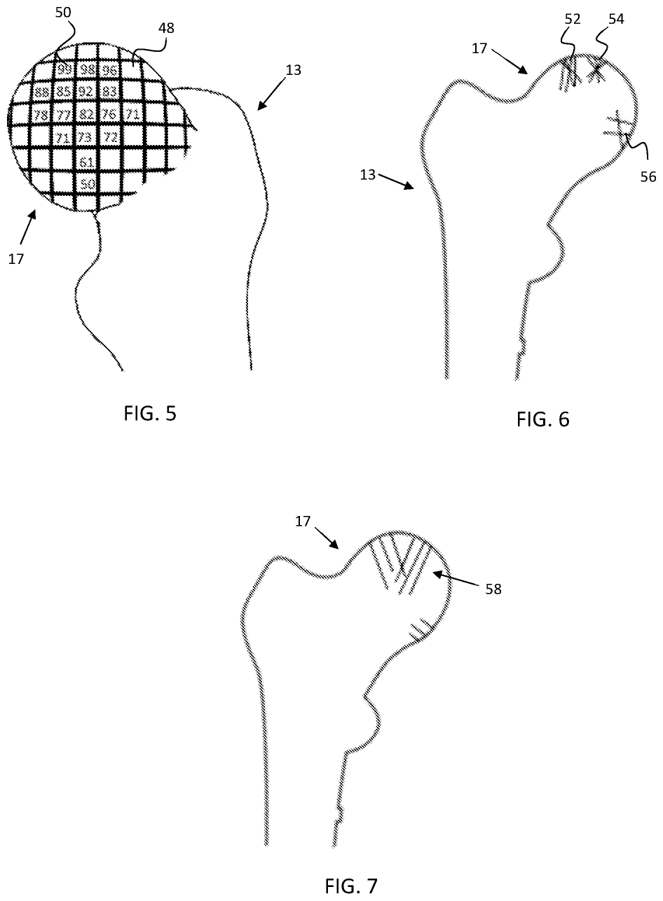

FIGS. 5, 6, and 7 depict a method for determining a force profile on femoral head bone data 17, which uses the same technique with respect to the acetabulum as described above. FIG. 5 depicts a plurality of voxels and the HUs representing density values of the bone at different locations on the femoral head bone data 17. FIG. 6 depicts the bone architecture at various locations (52, 54, and 56) on the femoral head bone data 17. FIG. 7 depicts a calculated force profile on the femoral head bone data 17 shown as a series of lines 58, where the magnitude of the lines correlates to the magnitude of the imposed forces.

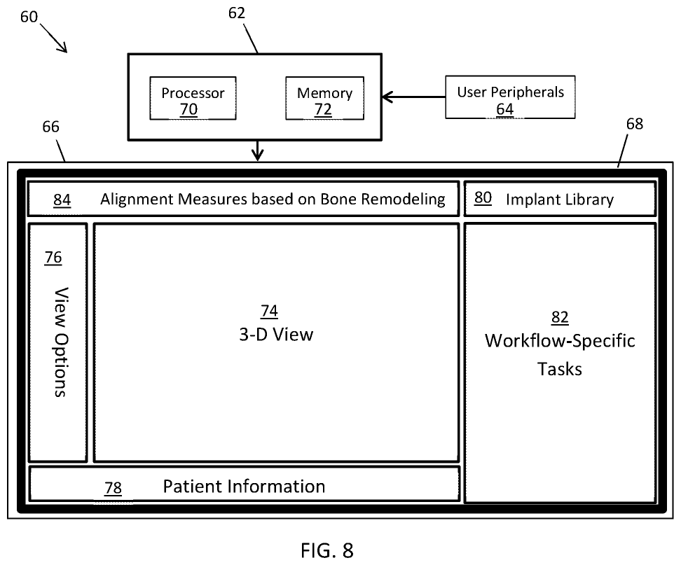

FIG. 8 depicts an embodiment of a planning workstation 60 for implementing embodiments of the invention. The workstation 60 includes a computer 62, user-peripherals 64, and a monitor 66 displaying a graphical user interface (GUI) 68. The computer 62 includes a processor 70 operatively coupled to non-transient memory 72 programmed with operating planning software to execute the planning methods described herein. The user peripherals 64 allow a user to interact with the GUI 68 and may include user input mechanisms such as a keyboard and mouse, or the monitor 66 may have touchscreen capabilities. The GUI 68 may include a three-dimensional (3-D) view window 74, a view options window 76, a patient information window 78, an implant library window 80, a workflow-specific tasks window 82, and an alignment measures window 84 for the position of the implant relative to the force profile indicative of the bone remodeling. Each window of the GUI 68 window can be summarized as follows. The 3-D view window 74 allows the user to view and interact with images (e.g., 3-D bone models, 3-D implant models). The view options window 76 provides options to change the view of the images, anatomical landmarks, or anatomical references (e.g., mechanical axes, distal condylar plane, posterior condylar plane, tibial plateau plane), select different views, and/or select the type of data displayed (e.g., display the force profile as a series of lines or as a heat map). The patient information window 78 displays the patient's information such as name, identification number, gender, surgical procedure, and operating side (e.g., left femur, right femur). The implant library window 80 may provide a drop-down menu to allow the user, or planning software, to select a particular implant (e.g., manufacturer A's implant in implant size 2) from a library of implants, and upon selection an implant image of the selected implant is displayed in the 3-D view window 74. The workflow-specific tasks window 82 includes various options to provide several functions illustratively including: guiding the user throughout different stages of the planning procedure; providing an option to switch between standard clinical alignment goals and force profile derived alignment; providing tools to adjust the POSE of an implant image with respect to a bone image in desired clinical directions (e.g., medial-lateral direction, flexion-extension rotation, proximal-distal direction); and displaying a summary of the surgical plan. The alignment measures window 84 displays the measured values of the alignment and position of the implant image with respect to the bone image (e.g., implant image is in 40° inclination and 20° anteversion). It should be appreciated that not all of the above windows be present in the planning software or viewable on the GUI 68.

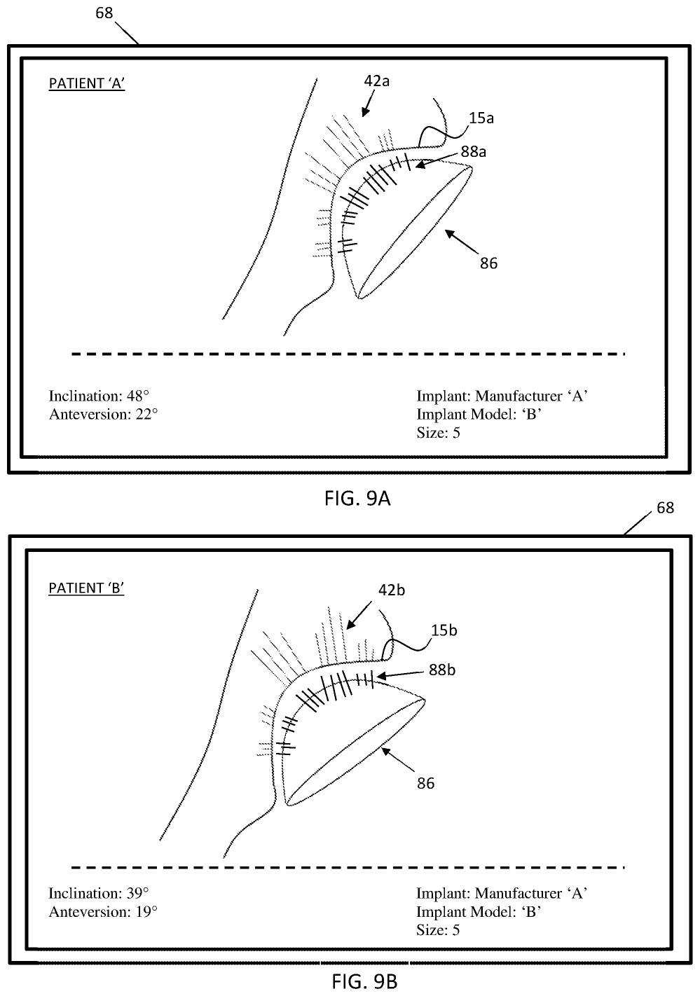

FIG. 9A depicts a surgical plan for patient ‘A’. The surgical plan is displayed on the GUI 68 and depicts acetabulum bone data 14a, the calculated force profile 42a, and image data (e.g., a 3-D implant model) of a cup implant 86 (i.e., cup implant image data 86). In a particular embodiment, the surgical plan is generated automatically by the software to position the cup implant image data 86 with respect to the acetabulum bone data 14a such that the forces imposed between the re-surfaced acetabulum and the cup implant mounted thereto, following the THA procedure, will substantially match the force profile 42a determined from the bone density data and bone architecture data. The software may automatically select an implant size, implant manufacturer, and implant model. In other embodiments, the user may manually position the cup implant image data 86 with respect to the acetabulum bone data 15 using software tools, or the software may position the cup implant image data 86 automatically, and then permit a user to adjust the position of the cup implant image data 86. An expected force profile 88a may be calculated to determine the expected forces imposed on the cup implant mounted to the re-surfaced acetabulum for different cup implant image data 86 positions. In other words, the expected force profile 88a is calculated and updated depending on the position of the cup implant image data 86 with respect to the acetabulum bone data 15a to show the expected forces imposed on the cup implant 86 following the procedure (e.g., in-vivo forces). The software, or the user, may position the cup implant image data 86 with respect to the acetabulum bone data 15a to obtain a similar force profile 88a on the cup implant as the force profile 42a on the acetabulum. The expected force profile 88a may be calculated using finite element analysis or machine learning, which may further incorporated the planned position for the femoral implant model with respect to a femoral bone model into the equation.

In another embodiment, the geometry of the cup implant image data 86 alone may be used to position the cup implant image data 86 with respect to the acetabulum bone data 15 according to the calculated force profile 42 from the bone density data and bone architecture data. For example, the orientation of the cup implant image data 86 may be adjusted in inclination or anteversion such that more of the cup implant image data 86 overlaps with areas of greater calculated forces. This is shown in FIG. 9A vs. FIG. 9B. Patient ‘B’ has greater forces near the top of the acetabulum compared to patient ‘A’. The cup implant image data 86 may therefore be positioned with less inclination such that more surface area of the cup implant image data 86 overlaps the areas of greater calculated forces.

It should be appreciated, that the magnitude and locations of the calculated forces in the force profiles 44 may be more retrograde or antegrade as well as more superior or less superior with respect to the bone, which allows for both the inclination angle and anteversion angle for a cup implant to be determined with respect to the acetabulum. In other words, the magnitudes and locations of the forces are calculated in three-dimensions around the surface of the bone. This allows the planning software and/or user to compare the forces in all directions and locations around the surface of the bone to determine an optimal location for an implant with respect to the bone.

It should be appreciated that a position for a femoral implant may be determined in a similar manner using the femoral bone data 13, a calculated force profile 58, and femoral implant image data (not shown). In particular embodiments, both the femoral bone data and acetabulum bone data are used in the calculations of at least one of: (i) a force profiles associated with the bone data; (ii) an expected force profile on the implant; and/or (iii) to determine a position for one or more implants with respect to one or more bones. The end result is a surgical plan tailored to the natural motion of the patient's joint as determined from the natural remodeling of the patient's bone. FIG. 9A depicts the final implant positioned in 48° inclination and 22° anteversion for patient ‘A’, and FIG. 9B depicts the final implant positioned in 39° inclination and 19° anteversion for patient ‘B’, both patient's set to receive the same type of implant (i.e., the same implant model and implant size. This is vastly different from conventional planning methods that use standard clinical alignment goals (e.g., Lewinnek safe zone) and may result in better clinical outcomes.

In certain cases, the patient's bone(s) in need of a joint replacement may be severely diseased (e.g., arthritic) with significant bone erosion. In these cases, the patient's non-diseased contra-lateral joint may be used to determine normal loading on the bones of the joint for the patient. For example, a patient may have a severely eroded left hip joint that needs a hip replacement. Since the bone(s) are severely eroded, the bone density data and bone architecture data of those bones are not reflective of the healthy, or even slightly diseased, state of the bones and therefore should not be used to calculate the aforementioned force profiles to aid in surgical planning. Any calculated force profile would not represent the pre-diseased motion of the patient's joint. To remedy this problem, the contra-lateral hip joint (i.e., the right hip joint in this example), may be used. A force profile on the bone(s) is calculated using the bone density data and bone architecture data from the bones in the right hip joint. This force profile may closely approximate, or match, the forces that were experienced on the bones of the left hip joint before the left bones became severely diseased. The calculated force profile associated with the bones of the contra-lateral right hip may be mapped to the eroded bones of the left hip to plan the placement for the implants with respect to the force profile as described above. In this manner, the forces and motions of the left hip joint may be restored to a native state (i.e., before the bones were severely diseased).

In other cases where a patient presents with a severely diseased bone, a force profile for the diseased bone may be calculated to determine how the bone remodeled as a result of the disease, which may assist in planning the placement for the implant. For a given bone, bone density data and bone microarchitecture data may be used to calculate a force profile across the bone as described above. The force profiles may indicate regions of non-diseased bone and regions of diseased bone. For example, areas of low bone density may indicate osteoporotic bone and the resulting force profile calculated in this area may be less than what is normal. In these cases, there may be various methods to assist in planning the implant placement. In one embodiment, the software, or the user, may position the implant relative to the bone to restore the area of osteoporotic bone to a more normal force profile. The normal force profile may be based on historical data (e.g., force profiles calculated for non-diseased bones of previous patients). The historical data may include averages (e.g., a force average across a population), standard deviations, and other statistical correlations. The software may then display an overlay of the calculated force profile compared to a normal force profile, which may be region based (e.g., osteoporotic regions) or for the entire portion of the bone being replaced. The software, or the user, may then adjust the position of the implant relative to the bone that would restore the forces experienced on the bone to a more normal force. This allows the software, or the user, to plan the placement for the implant relative to the bone that also accounts for the severity of the diseased bone.

In other embodiments, machine learning is used to train a model using one or more of the following inputs from historical data: bone density data; bone architecture data (e.g., patterns in the architecture data); force magnitudes; force vectors; patterns of the force profiles; bone geometry; patient demographic data (e.g., male or female, BMI, height, ethnicity); final implant placement relative to the bone; and clinical outcomes (e.g., pain scores, revision, recovery). The trained model then receives one or more of the following inputs of the bone for the present patient: bone density data; bone architecture data (e.g., patterns in the architecture data); force magnitudes; force vectors; patterns of the force profiles; and bone geometry. The output of the trained model may include one or more of: the severity and locations of the diseased bone; matching force profiles and how the force profiles compare to a patient population; and proposed locations for placing the implant relative to the bone that results in good clinical outcomes (e.g., restores the forces experienced on the bone to a more normal force). The trained model may determine an optimal location for an implant relative to a bone based on a bone's particular force profile, whether or not the bone is diseased. For example, the trained model may determine that a procedure for a bone with a particular force profile will have better clinical outcomes if the implant is adjusted in a particular direction (e.g., anteverted by 2 degrees) or by a certain amount (adjust implant 2 mm in the superior direction) relative to the classic implant placement targets.

### OTHER EMBODIMENTS

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient roadmap for implementing the exemplary embodiment or exemplary embodiments. It should be understood that various changes may be made in the function and arrangement of elements without departing from the scope as set forth in the appended claims and the legal equivalents thereof.

1. A computer-assisted method for determining a placement position for an implant relative to a bone, said method comprising:
calculating a force profile exerted on a bone from bone density data and bone architecture data; and
determining a position and orientation (POSE) for an implant in relation to the bone based, at least in part, on the calculated force profile.

2. The method of claim 1 wherein the bone density data and bone architecture data is obtained from computed tomography (CT) or magnetic resonance imaging (MRI).

3. The method of claim 1 wherein the force profile is calculated using at least one of finite element analysis or machine learning.

4. The method of claim 1 wherein the force profile is displayed via a graphical user interface (GUI).

5. The method of claim 4 wherein the force profile is displayed as one of a series of lines, a heat map, a matrix of values, or other graphical technique on a bone model.

6. The method of claim 1 wherein the force profile is viewed in two-dimensions (2-D) or three-dimensions (3-D) on a bone model.

7. The method of claim 1 wherein the force profile indicates how the bone remodeled under loading conditions for a joint and is indicative of a natural motion of the joint.

8. The method of claim 1 wherein the joint is one of a hip, knee, shoulder, or elbow.

9. The method of claim 1 wherein the determining of the position and orientation for the implant in relation to the bone comprises receiving computer input from a user to adjust a position or orientation of the implant in relation to the bone.

10. The method of claim 1 wherein the determining of the position and orientation for the implant in relation to the bone comprises determining a POSE for implant image data in relation to bone data.

11. A computing system, comprising:
a computer having a processor and non-transient memory programmed with operating planning software that when executed by the processor is configured to:
calculate a force profile exerted on a bone from bone density data and bone architecture data; and
determine a position and orientation (POSE) for an implant in relation to the bone based, at least in part, on the calculated force profile.

12. The system of claim 11 further comprising a graphical user interface (GUI).

13. The system of claim 12 wherein the GUI displays the calculated force profile with respect to bone data of the bone.

14. The system of claim 11 wherein the non-transient memory further stores implant image data for a plurality of different implants.

15. The system of claim 11 wherein the bone density data and bone architecture data is obtained from computed tomography (CT) or magnetic resonance imaging (MRI).

16. The system of claim 13 wherein the force profile is calculated using at least one of finite element analysis or machine learning.

17. The system of claim 13 wherein the determining of the position and orientation for the implant in relation to the bone comprises determining a POSE for implant image data in relation to bone data.

18. The system of claim 17 where the implant image data is a three-dimensional (3-D) model of the implant and the bone data is a 3-D model of the bone.

19. The system of claim 11 wherein the processor is configured to receive computer input from a user, wherein the position and orientation (POSE) for the implant in relation to the bone is determined using the computer input.

20. The system of claim 11 wherein the force profile indicates how the bone remodeled under loading conditions for a joint and is indicative of a natural motion of the joint.
